(19) United States
(12) Patent Application Publication
(10) Pub. No.: US 2003/0060983 A1
(43) Pub. Date: Mar. 27, 2003

(54) Proteomic analysis

(76) Inventor: Joseph Michael Daniel Figeys, Pickering (CA)

Correspondence Address: ROPES & GRAY, ONE INTERNATIONAL PLACE, BOSTON, MA, 02110-2624, US

(21) Appl. No.: 10/167,224

(22) Filed: Jun. 11, 2002

Related U.S. Application Data
(60) Provisional application No. 60/297,574, filed on Jun. 12, 2001.

(51) Int. Cl. G01N 33/00

(52) U.S. Cl. 702/28; 436/86; 436/173

(57) ABSTRACT

The instant invention relates to improved methods and systems for proteomic analysis using MS (mass spectrometry) data, particularly tandem mass spectrometry (MS/MS) data. The method comprises searching a first level database using sequence information generated by MS/MS obtained from a peptide selected from a digested protein sample. A second level database (which is smaller and contains only the candidate proteins) is then searched to identify the identity of the protein containing the selected peptide. More levels of even smaller databases can be generated for successive rounds of searches if the previous round of search does not unequivocally identify the protein containing the selected peptide(s). The method may be carried out on a range of mass spectrometers including a tandem mass spectrometer (MS/MS), an ion trap mass spectrometer or others capable of generating MS and MS/MS data.

### REFERENCE TO RELATED APPLICATIONS

This application claims priority to U.S. Provisional Application 60/297,574, filed on Jun. 12, 2001, the entire content of which is hereby incorporated by reference.

### FIELD OF THE INVENTION

The present invention relates to methods of proteomic analysis.

### BACKGROUND OF THE INVENTION

The following references arc incorporated herein by reference:

U.S. Pat. No. 6,017,693 to Yates et al.

U.S. Pat. No. 5,885,841 to Higgs et al.

International Application PCT/US98111157 to Li et al; and

International Application PCT/US99/12221 to Dancik.

Mass spectrometry has become an important tool in the identification of protein and other chemical analysis. With it, a researcher is able to identify protein, peptide or peptide fragments by comparing its mass spectrum data against proteins, DNA, and EST sequence databases. Several techniques are emerging to carry out that comparison. For example, U.S. Pat. No. 6,017,693, to Yates, et al discloses a system in which data is collected from a tandem mass spectrometer (MS/MS) to determine the mass of an unidentified peptide. A list of candidate sequences is collected from a protein sequence database or a nucleotide sequence database wherein each candidate has the same or (within a given tolerance level) similar mass to the unidentified peptide. The system then predicts the mass spectra for each candidate spectra and each is then compared against the mass spectral of the unidentified peptide using a closeness-of-fit measure.

The problem with procedures such as this is the size of the database being searched.

Yates contemplates the problem by suggesting that the initial list of candidates may be prefiltered according to a particular class of proteins, for example. Alternatively, the analysis may be restricted to some, rather than all, of the fragment ions in the MS/MS spectrum and those which are selected can be ranked. However, once the database is pre-filtered, the whole subsequence is used through out the analysis, which can still lead to relatively long processing times.

It is an object of the present invention to improve the speed of proteomic analysis.

### SUMMARY OF THE INVENTION

As used hereinbelow and in the claims, the term “MS data” is intended to mean mass information of peptides acquired by mass spectrometry. The term “MS/MS data” is intended to mean fragmentation patterns for an isolated peptide generated by mass spectrometry.

In one of its aspects, the present invention provides a method of analyzing a digested protein sample, comprising the steps of:

(a) generating an MS data set for the digested sample;

(b) selecting at least one peptide represented in the MS data set;

(c) generating an MS/MS data set for said at least one peptide selected in step (b);

(d) searching a first level protein database to find candidate proteins containing said at least one selected peptide(s);

(e) comparing MS data from the digested sample with one or more of the candidate proteins only, to find a match therebetween.

In this case, either the MS data collected in step (a), or another MS data set for the protein sample may be used in step (e).

In one embodiment, step (d) includes the step of:

(f) preparing a second level database containing only the candidate proteins, and searching the second level database to find candidate proteins containing said at least one selected peptide(s).

Alternatively, step (e) includes the step of:

(g) narrowing the search field in the first level protein database to search only the candidate proteins.

Preferably, the first level database includes digest data for each of the candidate proteins. However, as an alternative, in silico digest data may be prepared for one or more of the candidate proteins as the analysis progresses.

Preferably, step (e) includes the step of:

(i) selecting a peptide from the MS data set;

(j) searching the digest data for the candidate proteins to identify the selected peptide therein; and

(k) recording a match when the selected peptide is found in a candidate protein.

In another embodiment, step (e) also includes obtaining another MS data set for the protein sample.

Preferably, steps (i), (j), and (k) are repeated until a sufficient number of selected peptides are identified in a candidate protein to declare a match.

However, when a selected peptide of step (i) is not found in any one candidate protein, the method further includes the steps of:

(1) generating an MS/MS, data set for the selected peptide; and

(m) searching a first level protein database to find candidate proteins according to the selected peptide.

If desired, step (e) maybe conducted on one of a number of online databases such as those known by the trade names SEQQUEST, MASCOT or PROFOND or others. Alternatively, custom made databases may also be used, or a combination of the two.

The method may be carried out on a range of mass spectrometers including a tandem mass spectrometer (MS/MS), an ion trap mass spectrometer or others capable of generating MS and MS/MS data.

In another of its aspects, the present invention provides a method of analyzing a digested protein sample, comprising the steps of:

(a) generating an MS data set for the digested sample;

(b) selecting a first peptide represented in the data set;

(c) generating an MS/MS data set for the first selected peptide;

(d) searching at least one first level protein database to find at least one candidate protein which, by a predetermined measure, is identified to contain the first selected peptide;

(e) preparing a second level database containing only the candidate proteins of step (d);

(f) selecting a second peptide; and

(g) searching the second level database to find candidates which are identified to contain the selected second peptide; and wherein, if more than one candidate protein is identified in step (g), further comprising the steps of:

(h) selecting an n^{th} peptide, wherein n is preferably more than 3;

(i) searching the second level database to find candidates which are identified to contain the selected n^{th} peptide; and

(j) incrementing n and repeating steps (h), (i), until a single candidate protein is identified.

Preferably, step (e) includes the step of narrowing the search field in the first level database, or assembling a new second level database.

In another of its aspects, the present invention provides a protein analysis system, comprising:

(a) an MS unit for generating MS data on a digested protein sample;

(b) a selector unit for selecting a first peptide from the digested protein sample;

(c) an MS/MS unit for generating MS/MS data for the first peptide; and

(d) an identification unit for identifying the protein sample, the identification unit comprising:

(I) a search station operable in a first phase for searching at least one first level database to identify candidate proteins containing the first peptide;

(II) a memory station for storing at least one second level database containing only the candidate proteins;

(III) the search station being operable in a second phase to find a single target candidate protein by comparing the MS data front the digested protein sample with MS data for the candidate proteins.

In still another of its aspects, the present invention provides a protein analysis system, comprising:

(a) an MS unit for generating mass spectrum data on a digested protein sample;

(b) selection means for selecting a peptide from the digested protein sample;

(c) an MS/MS unit for generating mass spectrum data for the selected peptide; and

(d) an identification unit for identifying the protein sample, the identification unit comprising a general purpose computer programmed to carry out the steps of:

(I) searching at least one first level database to identify candidate proteins containing the first peptide;

(II) storing at least one second level database containing only the candidate proteins;

(III) searching second level database to identify a single target candidate protein by comparing the MS data from the digested protein sample with MS data for the candidate proteins.

In yet another of its aspects, the present invention provides a computer program product recorded on a computer-readable medium and including the computer executable steps of:

(a) initiating a computer data input to receive MS data of a digested protein sample;

(b) selecting one peptide from the MS data;

(c) initiating a computer data input to receive MS/MS data of the selected peptide;

(d) initiating a search of a protein database to find candidate proteins which, by some measure of confidence, contain the selected peptide;

(e) comparing the peptides of the digested protein with the candidate proteins in order to identify a candidate sharing a sufficient predetermined number of peptides to declare a match; and

(f) generating an output to report the match.

In yet another of its aspects, the present invention provides a method of protein analysis, comprising the steps of:

(a) selecting a peptide from MS data for a digested protein sample;

(b) recording MS/MS data for the selected peptide;

(c) initiating a search of a protein database to find candidate proteins which, by some measure of confidence, contain the selected peptide; and

(d) iteratively comparing the peptides of the digested protein with the candidate proteins in order to identify a candidate sharing a sufficient predetermined number of peptides to declare a match.

In another of its aspects, the present invention provides a method of protein analysis, comprising:

(a) preparing a sample comprising at least one unknown protein;

(b) adding to the sample at least one bait molecule;

(c) subjecting the baited sample to the method as defined hereinabove, wherein before step (c), the method includes the step of building a binding protein database according to proteins known to hind with the bait molecule or a consequential molecule thereof.

Preferably, step (c) includes the steps of:

(d) assembling a list of proteins known to bind with the bait molecule or a consequential molecule thereof;

(e) conducting an in silico digestion of the list of proteins to form the binding protein database.

In still another of its aspects, the present invention provides a method of protein analysis, comprising:

(a) preparing a list of known proteins and conducting an in silico digestion of the list of proteins to form a peptide database;

(b) providing a digested protein sample;

(c) recording MS data for the digested protein sample;

(d) selecting a first peptide from the digested protein sample;

(e) recording MS/MS data for the first selected peptide;

(f) initiating a search in the peptide database to find candidate proteins which, by a predetermined confidence value, contain the first selected peptide;

(g) selecting a second peptide; and

(h) comparing the MS data of the second peptide with the candidate proteins in order to find candidate proteins which contain both the first and second selected peptides.

Preferably, when more than one match has been found in step (h), the method further comprising the step of:

(i) selecting another peptide and repeating step (h).

Preferably, when a match is not found in step (h), the method further comprising the steps of:

(j) recording MS/MS data for the second selected peptide;

(k) initiating a search in the peptide database to find candidate proteins which, by a predetermined confidence value, contain the second selected peptide.

Thus, the second level database may simply involve the narrowing of the search fields for the search of the first level database. The second level database may not be digested in the sense of containing MS data or MS/MS data for the protein contained in it. In addition, the second level database may or may not be mass ordered. In the cases where the second level database is not digested and not mass ordered, such steps may be undertaken as desired and as needed during the analysis.

It is also contemplated that the method may be used to simultaneously spin-off parallel processes for residual masses that do not match to the second level databases. It may be appropriate in some cases to run parallel analyses of the MS data to find a match. In other words, masses that don't match to the second level databases may be used to continuously spin-off a new set of nth level databases, wherein the value n can be selected according to the particular analysis. This means that the depth at which the method “drills down” into a first level database, that is by refining a search field, can be controlled.

If desired, a range of information can be collected any information on the protein prior to analysis, which can be used to reduce the size of the database prior to any search. This includes information on protein interactions and protein functionality.

### BRIEF DESCRIPTION OF THE DRAWINGS

Several preferred embodiments of the present invention will now be described, by way of example only, with reference to the appended drawing in which:

FIG. 1 Schematic view of a system for proteomic analysis.

FIG. 2 Schematic views of an analytical method using the system of FIG. 1.

FIG. 3 Schematic views of an analytical method using the system of FIG. 1.

FIG. 4 Schematic views of an analytical method using the system of FIG. 1.

FIG. 5 Another schematic view of a system for proteomic analysis.

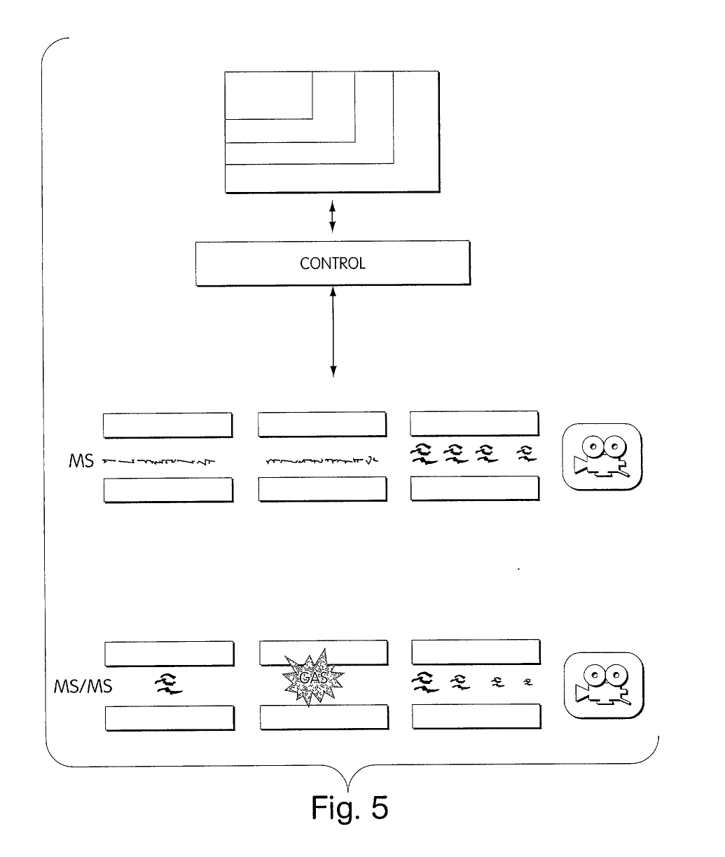

### DETAILED DESCRIPTION OF THE INVENTION

1. Overview

In one of its embodiments to be illustrated below, the present invention provides a novel approach to the handling of proteomic operations, termed “proteomic operating system,” devoted to direct all the operations using information extracted from protein-DNA databases. Examples of such databases are described in the above mentioned references.

2. Definitions

As used hereinbelow and in the claims, the term “MS data” is intended to mean mass information of peptides acquired by mass spectrometry. The term “MS/MS data” is intended to mean fragmentation patterns for an isolated peptide generated by mass spectrometry.

“Homology” or “identity” or “similarity” refers to sequence similarity between two peptides or between two nucleic acid molecules, with identity being a more strict comparison. Homology and identity can each be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the—sequences of the present invention.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both polypeptide and DNA databases.

Databases with individual sequences are described in Methods in Enzymology, ed. Doolittle, supra. Databases include Genbank, EMBL, and DNA Database of Japan (DDBJ). Preferred nucleic acids have a sequence at least 70%, and more preferably 80% identical and more preferably 90% and even more preferably at least 95% identical to an nucleic acid sequence of a sequence shown in one of SEQ ID Nos: 1-850. Nucleic acids at least 90%, more preferably 95%, and most preferably at least about 98-99% identical with a nucleic sequence represented in one of SEQ ID Nos: 1-4 are of course also within the scope of the invention. In preferred embodiments, the nucleic acid is mammalian. In comparing a new nucleic acid with known sequences, several alignment tools are available. Examples include PileUp, which creates a multiple sequence alignment, and is described in Feng et al., J. Mol. Evol. (1987) 25:351-360. Another method, GAP, uses the alignment method of Needleman et al., J. Mol. Biol. (1970) 48:443-453. GAP is best suited for global alignment of sequences. A third method, BestFit, functions by inserting gaps to maximize the number of matches using the local homology algorithm of Smith and Waterman, Adv. Appl. Math. (1981) 2:482-489.

The terms “protein”, “polypeptide” and “peptide” are used interchangeably herein when referring to a natural or recombinant gene product of fragment thereof.

The term “recombinant protein” refers to a polypeptide of the present invention which is produced by recombinant DNA techniques, wherein generally, DNA encoding a polypeptide is inserted into a suitable expression vector which is in turn used to transform a host cell to produce the heterologous polypeptide. Moreover, the phrase “derived from”, with respect to a recombinant gene, is meant to include within the meaning of “recombinant protein” those polypeptides having an amino acid sequence of a native polypeptide, or an amino acid sequence similar thereto which is generated by mutations including substitutions and deletions (including truncation) of a naturally occurring form of the polypeptide.

3. Mass Spectrometers and Detection Methods

### Mass Spectrometry

Mass spectrometry, also called mass spectroscopy, is an instrumental approach that allows for the gas phase generation of ions as well as their separation and detection. The five basic parts of any mass spectrometer include: a vacuum system; a sample introduction device; an ionization source; a mass analyzer; and an ion detector. A mass spectrometer determines the molecular weight of chemical compounds by ionizing, separating, and measuring molecular ions according to their mass-to-charge ratio (m/z). The ions are generated in the ionization source by inducing either the loss or the gain of a charge (e.g. electron ejection, protonation, or deprotonation). Once the ions are formed in the gas phase they can be electrostatically directed into a mass analyzer, separated according to mass and finally detected. The result of ionization, ion separation, and detection is a mass spectrum that can provide molecular weight or even structural information.

A common requirement of all mass spectrometers is a vacuum. A vacuum is necessary to permit ions to reach the detector without colliding with other gaseous molecules. Such collisions would reduce the resolution and sensitivity of the instrument by increasing the kinetic energy distribution of the ion's inducing fragmentation, or preventing the ions from reaching the detector. In general, maintaining a high vacuum is crucial to obtaining high quality spectra.

The sample inlet is the interface between the sample and the mass spectrometer. One approach to introducing sample is by placing a sample on a probe which is then inserted, usually through a vacuum lock, into the ionization region of the mass spectrometer. The sample can then be heated to facilitate thermal desorption or undergo any number of high-energy desorption processes used to achieve vaporization and ionization.

Capillary infusion is often used in sample introduction because it can efficiently introduce small quantities of a sample into a mass spectrometer without destroying the vacuum. Capillary columns are routinely used to interface the ionization source of a mass spectrometer with other separation techniques including gas chromatography (GC) and liquid chromatography (LC). Gas chromatography and liquid chromatography can serve to separate a solution into its different components prior to mass analysis. Prior to the 1980's, interfacing liquid chromatography with the available ionization techniques was unsuitable because of the low sample concentrations and relatively high flow rates of liquid chromatography. However, new ionization techniques such as electrospray were developed that now allow LC/MS to be routinely performed. One variation of the technique is that high performance liquid chromatography (HPLC) can now be directly coupled to mass spectrometer for integrated sample separation/preparation and mass spectrometer analysis.

In terms of sample ionization, two of the most recent techniques developed in the mid 1980's have had a significant impact on the capabilities of Mass Spectrometry: Electrospray Ionization (ESI) and Matrix Assisted Laser Desorption/Ionization (MALDI). ESI is the production of highly charged droplets which are treated with dry gas or heat to facilitate evaporation leaving the ions in the gas phase. MALDI uses a laser to desorb sample molecules from a solid or liquid matrix containing a highly UV-absorbing substance.

The MALDI-MS technique is based on the discovery in the late 1980s that an analyte consisting of, for example, large nonvolatile molecules such as proteins, embedded in a solid or crystalline “matrix” of laser light-absorbing molecules can be desorbed by laser irradiation and ionized from the solid phase into the gaseous or vapor phase, and accelerated as intact molecular ions towards a detector of a mass spectrometer. The “matrix” is typically a small organic acid mixed in solution with the analyte in a 10,000:1 molar ratio of matrix/analyte. The matrix solution can be adjusted to neutral pH before mixing with the analyte.

The MALDI ionization surface may be composed of an inert material or else modified to actively capture an analyte. For example, an analyte binding partner may be bound to the surface to selectively absorb a target analyte or the surface may be coated with a thin nitrocellulose film for nonselective binding to the analyte. The surface may also be used as a reaction zone upon which the analyte is chemically modified, e.g., CNBr degradation of protein. See Bai et al, Anal. Chem. 67, 1705-1710 (1995).

Metals such as gold, copper and stainless steel are typically used to form MALDI ionization surfaces. However, other commercially-available inert materials (e.g., glass, silica, nylon and other synthetic polymers, agarose and other carbohydrate polymers, and plastics) can be used where it is desired to use the surface as a capture region or reaction zone. The use of Nation and nitrocellulose-coated MALDI probes for on-probe purification of PCR-amplified gene sequences is described by Liu et al., Rapid Commun. Mass Spec. 9:735-743 (1995). Tang et al. have reported the attachment of purified oligonucleotides to beads, the tethering of beads to a probe element, and the use of this technique to capture a complimentary DNA sequence for analysis by MALDI-TOF MS (reported by K. Tang et al., at the May 1995 TOF-MS workshop, R. J. Cotter (Chairperson); K. Tang et al., Nucleic Acids Res. 23, 3126-3131, 1995). Alternatively, the MALDI surface may be electrically- or magnetically activated to capture charged analytes and analytes anchored to magnetic beads respectively.

Aside from MALDI, Electrospray Ionization Mass Spectrometry (ESI/MS) has been recognized as a significant tool used in the study of proteins, protein complexes and bi-omolecules in general. ESI is a method of sample introduction for mass spectrometric analysis whereby ions are formed at atmospheric pressure and then introduced into a mass spectrometer using a special interface. Large organic molecules, of molecular weight over 10,000 Daltons, may be analyzed in a quadrupole mass spectrometer using ESI.

In ESI, a sample solution containing molecules of interest and a solvent is pumped into an electrospray chamber through a fine needle. An electrical potential of several kilovolts may be applied to the needle for generating a fine spray of charged droplets. The droplets may be sprayed at atmospheric pressure into a chamber containing a heated gas to vaporize the solvent. Alternatively, the needle may extend into an evacuated chamber, and the sprayed droplets are then heated in the evacuated chamber. The fine spray of highly charged droplets releases molecular ions as the droplets vaporize at atmospheric pressure. In either case, ions are focused into a beam, which is accelerated by an electric field, and then analyzed in a mass spectrometer.

Because electrospray ionization occurs directly from solution at atmospheric pressure, the ions formed in this process tend to be strongly solvated. To carry out meaningful mass measurements, solvent molecules attached to the ions should be efficiently removed, that is, the molecules of interest should be “desolvated.” Desolvation can, for example, be achieved by interacting the droplets and solvated ions with a strong countercurrent flow (6-9 l/m) of a heated gas before the ions enter into the vacuum of the mass analyzer.

Other well-known ionization methods may also be used. For example, electron ionization (also known as electron bombardment and electron impact), atmospheric pressure chemical ionization (APCI), fast atom Bombardment (FAB), or chemical ionization (CI).

Immediately following ionization, gas phase ions enter a region of the mass spectrometer known as the mass analyzer. The mass analyzer is used to separate ions within a selected range of mass to charge ratios. This is an important part of the instrument because it plays a large role in the instrument's accuracy and mass range. Ions are typically separated by magnetic fields, electric fields, and/or measurement of the time an ion takes to travel a fixed distance.

If all ions with the same charge enter a magnetic field with identical kinetic energies a definite velocity will be associated with each mass and the radius will depend on the mass. Thus a magnetic field can be used to separate a monoenergetic ion beam into its various mass components. Magnetic fields will also cause ions to form fragment ions. If there is no kinetic energy of separation of the fragments the two fragments will continue along the direction of motion with unchanged velocity. Generally, some kinetic energy is lost during the fragmentation process creating noninteger mass peak signals which can be easily identified. Thus, the action of the magnetic field on fragmented ions can be used to give information on the individual fragmentation processes taking place in the mass spectrometer.

Electrostatic fields exert radial forces on ions attracting them towards a common center. The radius of an ion's trajectory will be proportional to the ion's kinetic energy as it travels through the electrostatic field. Thus an electric field can be used to separate ions by selecting for ions that travel within a specific range of radii which is based on the kinetic energy and is also proportion to the mass of each ion.

Quadrupole mass analyzers have been used in conjunction with electron ionization sources since the 1950s. Quadrupoles are four precisely parallel rods with a direct current (DC) voltage and a superimposed radio-frequency (RF) potential. The field on the quadrupoles determines which ions are allowed to reach the detector. The quadrupoles thus function as a mass filter. As the field is imposed, ions moving into this field region will oscillate depending on their mass-to-charge ratio and, depending on the radio frequency field, only ions of a particular m/z can pass through the filter. The m/z of an ion is therefore determined by correlating the field applied to the quadrupoles with the ion reaching the detector. A mass spectrum can be obtained by scanning the RF field. Only ions of a particular m/z are allowed to pass through.

Electron ionization coupled with quadrupole mass analyzers can be employed in practicing the instant invention. Quadrupole mass analyzers have found new utility in their capacity to interface with electrospray ionization. This interface has three primary advantages. First, quadrupoles are tolerant of relatively poor vacuums (˜5×10⁻⁵ torr), which makes it well-suited to electrospray ionization since the ions are produced under atmospheric pressure conditions. Secondly, quadrupoles are now capable of routinely analyzing up to an m/z of 3000, which is useful because electrospray ionization of proteins and other biomolecules commonly produces a charge distribution below m/z 3000. Finally, the relatively low cost of quadrupole mass spectrometers makes them attractive as electrospray analyzers.

The ion trap mass analyzer was conceived of at the same time as the quadrupole mass analyzer. The physics behind both of these analyzers is very similar. In an ion trap the ions are trapped in a radio frequency quadrupole field. One method of using an ion trap for mass spectrometry is to generate ions externally with ESI or MALDI, using ion optics for sample injection into the trapping volume. The quadrupole ion trap typically consist of a ring electrode and two hyperbolic endcap electrodes. The motion of the ions trapped by the electric field resulting from the application of RF and DC voltages allows ions to be trapped or ejected from the ion trap. In the normal mode the RF is scanned to higher voltages, the trapped ions with the lowest m/z and are ejected through small holes in the endcap to a detector (a mass spectrum is obtained by resonantly exciting the ions and thereby ejecting from the trap and detecting them). As the RF is scanned further, higher m/z ratios become are ejected and detected. It is also possible to isolate one ion species by ejecting all others from the trap. The isolated ions can subsequently be fragmented by collisional activation and the fragments detected. The primary advantages of quadrupole ion traps is that multiple collision-induced dissociation experiments can be performed without having multiple analyzers. Other important advantages include its compact size, and the ability to trap and accumulate ions to increase the signal-to-noise ratio of a measurement.

Quadrupole ion traps can be used in conjunction with electrospray ionization MS/MS experiments in the instant invention.

The earliest mass analyzers separated ions with a magnetic field. In magnetic analysis, the ions are accelerated (using an electric field) and are passed into a magnetic field. A charged particle traveling at high speed passing through a magnetic field will experience a force, and travel in a circular motion with a radius depending upon the m/z and speed of the ion. A magnetic analyzer separates ions according to their radii of curvature, and therefore only ions of a given m/z will be able to reach a point detector at any given magnetic field. A primary limitation of typical magnetic analyzers is their relatively low resolution.

In order to improve resolution, single-sector magnetic instruments have been replaced with double-sector instruments by combining the magnetic mass analyzer with an electrostatic analyzer. The electric sector acts as a kinetic energy filter allowing only ions of a particular kinetic energy to pass through its field, irrespective of their mass-to-charge ratio. Given a radius of curvature, R, and a field, E, applied between two curved plates, the equation R=2V/E allows one to determine that only ions of energy V will be allowed to pass. Thus, the addition of an electric sector allows only ions of uniform kinetic energy to reach the detector, thereby increasing the resolution of the two sector instrument to 100,000. Magnetic double-focusing instrumentation is commonly used with FAB and EI ionization, however they are not widely used for electrospray and MALDI ionization sources primarily because of the much higher cost of these instruments. But in theory, they can be employed to practice the instant invention.

ESI and MALDI-MS commonly use quadrupole and time-of-flight mass analyzers, respectively. The limited resolution offered by time-of-flight mass analyzers, combined with adduct formation observed with MALDI-MS, results in accuracy on the order of 0.1% to a high of 0.01%, while ESI typically has an accuracy on the order of 0.01%. Both ESI and MALDI are now being coupled to higher resolution mass analyzers such as the ultrahigh resolution (>10⁵) mass analyzer. The result of increasing the resolving power of ESI and MALDI mass spectrometers is an increase in accuracy for biopolymer analysis.

Fourier-transform ion cyclotron resonance (FTMS) offers two distinct advantages, high resolution and the ability to tandem mass spectrometry experiments. FTMS is based on the principle of a charged particle orbiting in the presence of a magnetic field. While the ions are orbiting, a radio frequency (RF) signal is used to excite them and as a result of this RF excitation, the ions produce a detectable image current. The time-dependent image current can then be Fourier transformed to obtain the component frequencies of the different ions which correspond to their m/z.

Coupled to ESI and MALDI, FTMS offers high accuracy with errors as low as ±0.001%. The ability to distinguish individual isotopes of a protein of mass 29,000 is demonstrated.

A time-of-flight (TOF) analyzer is one of the simplest mass analyzing devices and is commonly used with MALDI ionization. Time-of-flight analysis is based on accelerating a set of ions to a detector with the same amount of energy. Because the ions have the same energy, yet a different mass, the ions reach the detector at different times. The smaller ions reach the detector first because of their greater velocity and the larger ions take longer, thus the analyzer is called time-of-flight because the mass is determine from the ions' time of arrival.

The arrival time of an ion at the detector is dependent upon the mass, charge, and kinetic energy of the ion. Since kinetic energy (KE) is equal to ½ mv² or velocity v=(2KE/m)^{½}, ions will travel a given distance, d, within a time, t, where t is dependent upon their m/z.

The magnetic double-focusing mass analyzer has two distinct parts, a magnetic sector and an electrostatic sector. The magnet serves to separate ions according to their mass-to-charge ratio since a moving charge passing through a magnetic field will experience a force, and travel in a circular motion with a radius of curvature depending upon the m/z of the ion. A magnetic analyzer separates ions according to their radii of curvature, and therefore only ions of a given m/z will be able to reach a point detector at any given magnetic field. A primary limitation of typical magnetic analyzers is their relatively low resolution. The electric sector acts as a kinetic energy filter allowing only ions of a particular kinetic energy to pass through its field, irrespective of their mass-to-charge ratio. Given a radius of curvature, R, and a field, E, applied between two curved plates, the equation R=2V/E allows one to determine that only ions of energy V will be allowed to pass. Thus, the addition of an electric sector allows only ions of uniform kinetic energy to reach the detector, thereby increasing the resolution of the two sector instrument.

The new ionization techniques are relatively gentle and do not produce a significant amount of fragment ions, this is in contrast to electron ionization (EI) which produces many fragment ions. To generate more information on the molecular ions generated in the ESI and MALDI ionization sources, it has been necessary to apply techniques such as tandem mass spectrometry (MS/MS), to induce fragmentation. Tandem mass spectrometry (abbreviated MSn—where n refers to the number of generations of fragment ions being analyzed) allows one to induce fragmentation and mass analyze the fragment ions. This is accomplished by collisionally generating fragments from a particular ion and then mass analyzing the fragment ions.

Fragmentation can be achieved by inducing ion/molecule collisions by a process known as collision-induced dissociation (CID) or also known as collision-activated dissociation (CAD). CID is accomplished by selecting an ion of interest with a mass filter/analyzer and introducing that ion into a collision cell. A collision gas (typically Ar, although other noble gases can also be used) is introduced into the collision cell, where the selected ion collides with the argon atoms, resulting in fragmentation. The fragments can then be analyzed to obtain a fragment ion spectrum. The abbreviation MSn is applied to processes which analyze beyond the initial fragment ions (MS2) to second (MS3) and third generation fragment ions (MS4). Tandem mass analysis is primarily used to obtain structural information, such as protein or polypeptide sequence, in the instant invention.

In certain instruments, such as those by JEOL USA, Inc. (Peabody, Mass.), the magnetic and electric sectors in any JEOL magnetic sector mass spectrometer can be scanned together in “linked scans” that provide powerful MS/MS capabilities without requiring additional mass analyzers. Linked scans can be used to obtain product-ion mass spectra, precursor-ion mass spectra, and constant neutral-loss mass spectra. These can provide structural information and selectivity even in the presence of chemical interferences. Constant neutral loss spectrum essentially “lifts out” only the interested peaks away from all the background peaks, hence removing the need for class separation and purification. Neutral loss spectrum can be routinely generated by a number of commercial mass spectrometer instruments (such as the one used in the Example section). JEOL mass spectrometers can also perform fast linked scans for GC/MS/MS and LC/MS/MS experiments.

Once the ion passes through the mass analyzer it is then detected by the ion detector, the final element of the mass spectrometer. The detector allows a mass spectrometer to generate a signal (current) from incident ions, by generating secondary electrons, which are further amplified. Alternatively some detectors operate by inducing a current generated by a moving charge. Among the detectors described, the electron multiplier and scintillation counter are probably the most commonly used and convert the kinetic energy of incident ions into a cascade of secondary electrons. Ion detection can typically employ Faraday Cup, Electron Multiplier, Photomultiplier Conversion Dynode (Scintillation Counting or Daly Detector), High-Energy Dynode Detector (HED), Array Detector, or Charge (or Inductive) Detector.

The introduction of computers for MS work entirely altered the manner in which mass spectrometry was performed. Once computers were interfaced with mass spectrometers it was possible to rapidly perform and save analyses. The introduction of faster processors and larger storage capacities has helped launch a new era in mass spectrometry. Automation is now possible allowing for thousands of samples to be analyzed in a single day. Te use of computer also helps to develop mass spectra databases which can be used to store experimental results. Software packages not only helped to make the mass spectrometer more user friendly but also greatly expanded the instrument's capabilities.

The ability to analyze complex mixtures has made MALDI and ESI very useful for the examination of proteolytic digests, an application otherwise known as protein mass mapping. Through the application of sequence specific proteases, protein mass mapping allows for the identification of protein primary structure. Performing mass analysis on the resulting proteolytic fragments thus yields information on fragment masses with accuracy approaching ±5 ppm, or ±0.005 Da for a 1,000 Da peptide. The protease fragmentation pattern is then compared with the patterns predicted for all proteins within a database and matches are statistically evaluated. Since the occurrence of Arg and Lys residues in proteins is statistically high, trypsin cleavage (specific for Arg and Lys) generally produces a large number of fragments which in turn offer a reasonable probability for unambiguously identifying the target protein.

The characterization of methylation status of a given polypeptide is extremely important for the study of PRMT and their functions in regulating a number of important biological cellular functions. Sometimes, the exact identity of a polypeptide being analyzed is not certain. In these situations, mass spectrometry has the added advantage of identifying polypeptide sequences containing the methylated arginine residue(s). The primary tools in these protein identification experiments are mass spectrometry, proteases, and computer-facilitated data analysis. As a result of generating intact ions, the molecular weight information on the peptides/proteins are quite unambiguous. Sequence specific enzymes can then provide protein fragments that can be associated with proteins within a database by correlating observed and predicted fragment masses. The success of this strategy, however, relies on the existence of the protein sequence within the database. With the availability of the human genome sequence (which indirectly contain the sequence information of all the proteins in the human body) and genome sequences of other organisms (mouse, rat, Drosophila, C. elegans, bacteria, yeasts, etc.), identification of the proteins can be quickly determined simply by measuring the mass of proteolytic fragments.

### Protease Digestion

One aspect of the instant invention is that peptide fragments ending with lysine or arginine residues can be used for sequencing with tandem mass spectrometry. While trypsin is the preferred the protease, many different enzymes can be used to perform the digestion to generate peptide fragments ending with Lys or Arg residues. For instance, in page 886 of a 1979 publication of Enzymes (Dixon, M. et al. ed., 3rd edition, Academic Press, New York and San Francisco, the content of which is incorporated herein by reference), a host of enzymes are listed which all have preferential cleavage sites of either Arg- or Lys- or both, including Trypsin [EC 3.4.21.4], Thrombin [EC 3.4.21.5], Plasmin [EC 3.4.21.7], Kallikrein [EC 3.4.21.8], Acrosin [EC 3.4.21.10], and Coagulation factor Xa [EC 3.4.21.6]. Particularly, Acrosin is the Trypsin-like enzyme of spermatoza, and it is not inhibited by α1-antitrypsin. Plasmin is cited to have higher selectivity than Trypsin, while Thrombin is said to be even more selective. However, this list of enzymes are for illustration purpose only and is not intended to be limiting in any way. Other enzymes known to reliably and predictably perform digestions to generate the polypeptide fragments as described in the instant invention are also within the scope of the invention.

### Sequence and Literature Databases and Database Search

The raw data of mass spectrometry will be compared to public, private or commercial databases to determine the identity of polypeptides.

BLAST search can be performed at the NCBI's (National Center for Biotechnology Information) BLAST website. According to the NCBI BLAST website, BLAST® (Basic Local Alignment Search Tool) is a set of similarity search programs designed to explore all of the available sequence databases regardless of whether the query is protein or DNA. The BLAST programs have been designed for speed, with a minimal sacrifice of sensitivity to distant sequence relationships. The scores assigned in a BLAST search have a well-defined statistical interpretation, making real matches easier to distinguish from random background hits. BLAST uses a heuristic algorithm which seeks local as opposed to global alignments and is therefore able to detect relationships among sequences which share only isolated regions of similarity (Altschul et al., 1990, J. Mol. Biol. 215: 403-10). The BLAST website also offer a “BLAST course,” which explains the basics of the BLAST algorithm, for a better understanding of BLAST.

For protein sequence search, several protein-protein BLAST can be used. Protein BLAST allows one to input protein sequences and compare these against other protein sequences. “Standard protein-protein BLAST” takes protein sequences in FASTA format, GenBank Accession numbers or GI numbers and compares them against the NCBI protein databases (see below).

“PSI-BLAST” (Position Specific Iterated BLAST) uses an iterative search in which sequences found in one round of searching are used to build a score model for the next round of searching. Highly conserved positions receive high scores and weakly conserved positions receive scores near zero. The profile is used to perform a second (etc.) BLAST search and the results of each “iteration” used to refine the profile. This iterative searching strategy results in increased sensitivity.

“PHI-BLAST” (Pattern Hit Initiated BLAST) combines matching of regular expression pattern with a Position Specific iterative protein search. PHI-BLAST can locate other protein sequences which both contain the regular expression pattern and are homologous to a query protein sequence.

“Search for short, nearly exact sequences” is an option similar to the standard protein-protein BLAST with the parameters set automatically to optimize for searching with short sequences. A short query is more likely to occur by chance in the database. Therefore increasing the Expect value threshold, and also lowering the word size is often necessary before results can be returned. Low Complexity filtering has also been removed since this filters out larger percentage of a short sequence, resulting in little or no query sequence remaining. Also for short protein sequence searches the Matrix is changed to PAM-30 which is better suited to finding short regions of high similarity.

The databases that can be searched by the BLAST program is user selected, and is subject to frequent updates at NCBI. The most commonly used ones are:

Nr: All non-redundant GenBank CDS translations+PDB+SwissProt+PIR+PRF;

Month: All new or revised GenBank CDS translation+PDB+SwissProt+PIR+PRF released in the last 30 days;

Swissprot: Last major release of the SWISS-PROT protein sequence database (no updates);

Drosophila genome: Drosophila genome proteins provided by Celera and Berkeley Drosophila Genome Project (BDGP);

S. cerevisiae: Yeast (Saccharomyces cerevisiae) genomic CDS translations;

Ecoli: Escherichia coli genomic CDS translations;

Pdb: Sequences derived from the 3-dimensional structure from Brookhaven Protein Data Bank;

Alu: Translations of select Alu repeats from REPBASE, suitable for masking Alu repeats from query sequences. It is available by anonymous FTP from the NCBI website. See “Alu alert” by Claverie and Makalowski, Nature vol. 371, page 752 (1994).

Some of the BLAST databases, like SwissProt, PDB and Kabat are complied outside of NCBI. Other like ecoli, dbEST and month, are subsets of the NCBI databases. Other “virtual Databases” can be created using the “Limit by Entrez Query” option.

The Welcome Trust Sanger Institute offer the Ensembl sofeware system which produces and maintains automatic annotation on eukaryotic genomes. All data and codes can be downloaded without constraints from the Sanger Centre website. The Centre also provides the Ensembl's International Protein Index databases which contain more than 90% of all known human protein sequences and additional prediction of about 10,000 proteins with supporting evidence. All these can be used for database search purposes.

In addition, many commercial databases are also available for search purposes. For example, Celera has sequenced the whole human genome and offers commercial access to its proprietary annotated sequence database (Discovery™ database).

Various softwares can be employed to search these databases. The probability search sofeware Mascot (Matrix Science Ltd.). Mascot utilizes the Mowse search algorithm and scores the hits using a probabilistic measure (Perkins et al., 1999, Electrophoresis 20: 3551-3567, the entire contents are incorporated herein by reference). The Mascot score is a function of the database utilized, and the score can be used to assess the null hypothesis that a particular match occurred by chance. Specifically, a Mascot score of 46 implies that the chance of a random hit is less than 5%. However, the total score consists of the individual peptide scores, and occasionally, a high total score can derive from many poor hits. To exclude this possibility, only “high quality” hits—those with a total score>46 with at least a single peptide match with a score of 30 ranking number 1—are considered.

Other similar softwares can also be used according to manufacturer's suggestion.

To determine if a particular protein is novel, that is, whether it is not previously found to localize to a particular subcellular compartment or organelle, further search of bioinformatics databases are necessary. One useful database for this type of literature search is PubMed.

PubMed, available via the NCBI Entrez retrieval system, was developed by the National Center for Biotechnology Information (NCBI) at the National Library of Medicine (NLM), located at the National Institutes of Health (NIH). The PubMed database was developed in conjunction with publishers of biomedical literature as a search tool for accessing literature citations and linking to full-text journal articles at web sites of participating publishers.

Publishers participating in PubMed electronically supply NLM with their citations prior to or at the time of publication. If the publisher has a web site that offers full-text of its journals, PubMed provides links to that site, as well as sites to other biological data, sequence centers, etc. User registration, a subscription fee, or some other type of fee may be required to access the full-text of articles in some journals.

In addition, PubMed provides a Batch Citation Matcher, which allows publishers (or other outside users) to match their citations to PubMed entries, using bibliographic information such as journal, volume, issue, page number, and year. This permits publishers easily to link from references in their published articles directly to entries in PubMed.

PubMed provides access to bibliographic information which includes MEDLINE as well as:

The out-of-scope citations (e.g., articles on plate tectonics or astrophysics) from certain MEDLINE journals, primarily general science and chemistry journals, for which the life sciences articles are indexed for MEDLINE.

Citations that precede the date that a journal was selected for MEDLINE indexing.

Some additional life science journals that submit full text to PubMed Central and receive a qualitative review by NLM.

PubMed also provides access and links to the integrated molecular biology databases included in NCBI's Entrez retrieval system. These databases contain DNA and protein sequences, 3-D protein structure data, population study data sets, and assemblies of complete genomes in an integrated system.

MEDLINE is the NLM's premier bibliographic database covering the fields of medicine, nursing, dentistry, veterinary medicine, the health care system, and the preclinical sciences. MEDLINE contains bibliographic citations and author abstracts from more than 4,300 biomedical journals published in the United States and 70 other countries. The file contains over 11 million citations dating back to the mid-1960's. Coverage is worldwide, but most records are from English-language sources or have English abstracts.

PubMed's in-process records provide basic citation information and abstracts before the citations are indexed with NLM's MeSH Terms and added to MEDLINE. New in process records are added to PubMed daily and display with the tag [PubMed—in process]. After MeSH terms, publication types, GenBank accession numbers, and other indexing data are added, the completed MEDLINE citations are added weekly to PubMed.

Citations received electronically from publishers appear in PubMed with the tag [PubMed—as supplied by publisher]. These citations are added to PubMed Tuesday through Saturday. Most of these progress to In Process, and later to MEDLINE status. Not all citations will be indexed for MEDLINE and are tagged, [PubMed—as supplied by publisher].

The Batch Citation Matcher allows users to match their own list of citations to PubMed entries, using bibliographic information such as journal, volume, issue, page number, and year. The Citation Matcher reports the corresponding PMID. This number can then be used to easily to link to PubMed. This service is frequently used by publishers or other database providers who wish to link from bibliographic references on their web sites directly to entries in PubMed.

### EXEMPLIFICATION

The invention now being generally described, it will be more readily understood by reference to the following examples which are included merely for purposes of illustration of certain aspects and embodiments of the present invention, and are not intended to limit the scope of the invention.

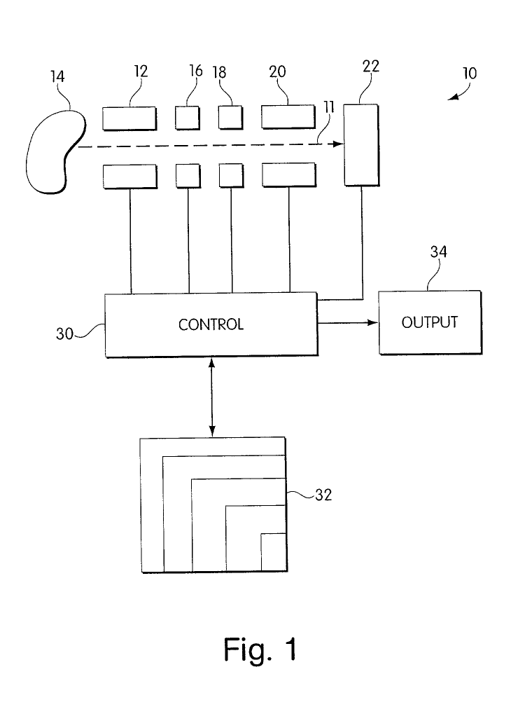

FIG. 1 illustrates, schematically, an exemplified protein analysis system (10) which includes a triple quadrupole mass spectrometer, it being understood that other mass spectrometers (such as those described above) may also be used which have different physical characteristics and methods of generating MS and MS/MS data. The system has a sample pathway (11), and a first MS unit (12) positioned on the pathway to receive a digested protein sample from the sample source (14). The sample source may include both a sample introduction device and an ionization source as described above. The first MS unit (12) separates the protein sample fragments by their mass. Downstream from the first MS unit (12) is a selection unit (16) for selecting a protein fragment for further spectral analysis. Typically, the selection unit (16) filters all but the selected peptide by selecting only those with a particular mass/charge ratio. Downstream from the selection unit (16) is a collision cell (18) in which the selected peptide is fragmented, and a second MS unit (20) for separating the peptide fragments according to their mass. The first MS unit (12), the selection unit (16), the collision cell (18), and the second MS unit (20) belongs to the mass analyzer section as described above. The protein sample fragments and the peptide fragments are detected by an ion detector (22).

A controller (30) communicates with each of the first MS unit (12), the sample source (14), the selection unit (16), the collision cell (18), the second MS unit (20) and the ion detector (22). As will be described, the controller also communicates with a database shown generally at (32) and, through a number of algorithms, compares MS or MS/MS data on a particular protein sample, peptide or peptide fragment with known protein data in the database in order to identify the protein. The controller communicates with an output shown at (32) to present the identity of the protein under investigation.

A particular feature of the device (10) is its ability to identify or analyze a protein sample by preparing a number of increasingly smaller databases, or by annotating the peptide entries related to a protein in such as manner as to highlight the identified protein, in order to reduce the overall period of time needed for an analysis. In other words, the system may be used to label or “tick” the peptides found in both the sample peptide and a candidate peptide, until the right protein is identified according to preset criteria.

The device functions as follows. First, the sample source (14) delivers a digested protein sample to the first MS unit (12), which separates the protein fragments by their mass as is known by those skilled in the art. The digested protein sample progresses through the device on the sample pathway (11) until its fragments register with the ion detector (22). The ion detector conveys MS/MS data to the controller which then selects a peptide for further analysis. The controller then conducts a search of the first level database (32) to find candidate proteins which either contain or are likely to contain the selected peptide. The controller then assembles a second level database containing only the candidate proteins.

The controller then begins an iterative task of identifying peptides and conducting a search of the second level database to find candidate proteins which contain the peptides from the first and second search and then assembles a base containing just them. This iteration continues as the number candidates is reduced.

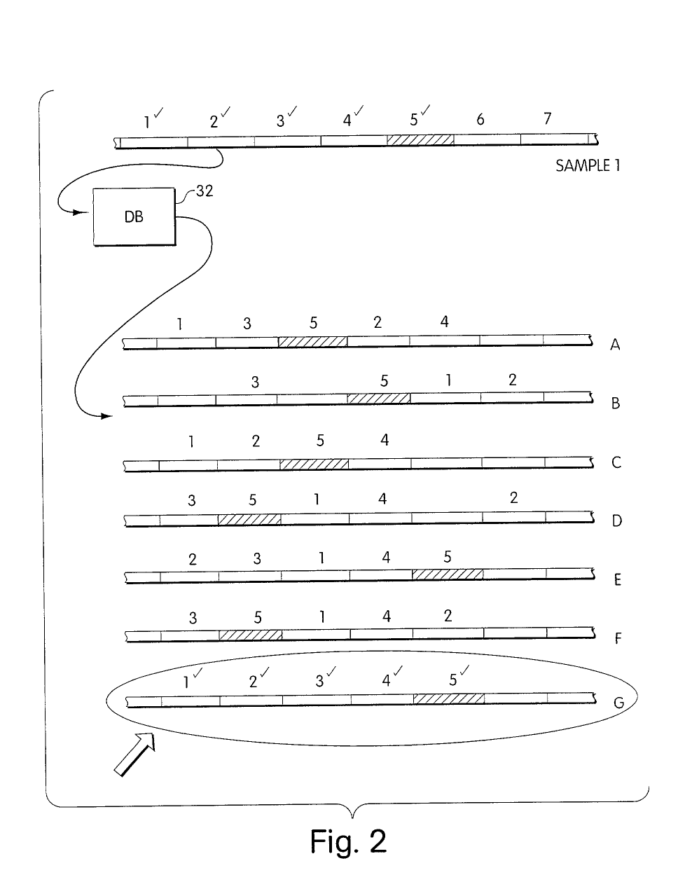

FIGS. 2, 3 and 4 illustrate the technique schematically. As shown in FIG. 2, a sample labeled as Sample 1 is delivered to the system which generates MS data indicating, in this example for the sake of illustration, seven peptides. One peptide, number 5, is selected (as shown by the hatched lines) and MS/MS data is generated for peptide 5. The system then searches the database and the result is, again for the sake of illustration, candidate proteins A to G, each shown to contain the peptide 5 (in dashed lines). The system then identified, one by one, peptides and checks them off against each of the candidate proteins. In this example, 5 peptides have been checked and a match is declared, namely with protein G.

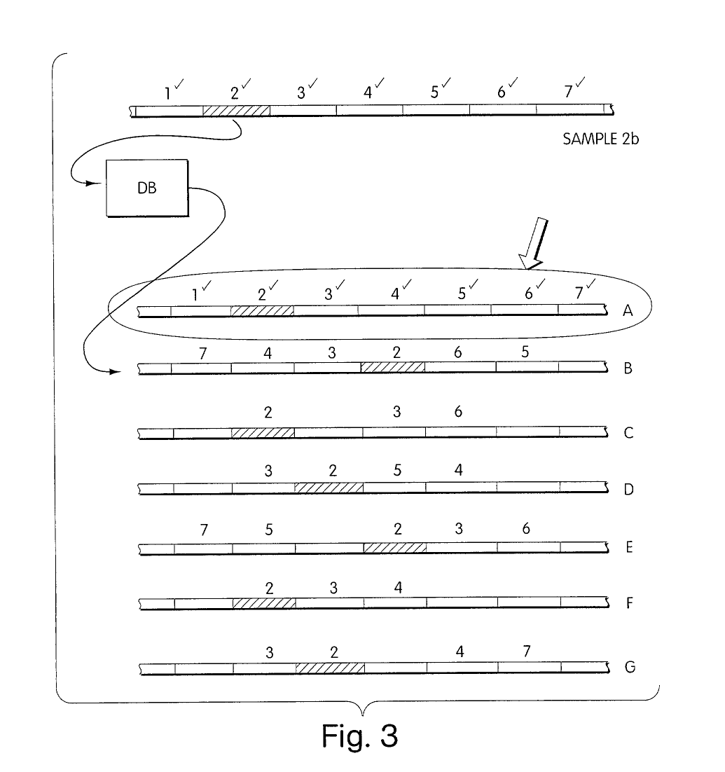

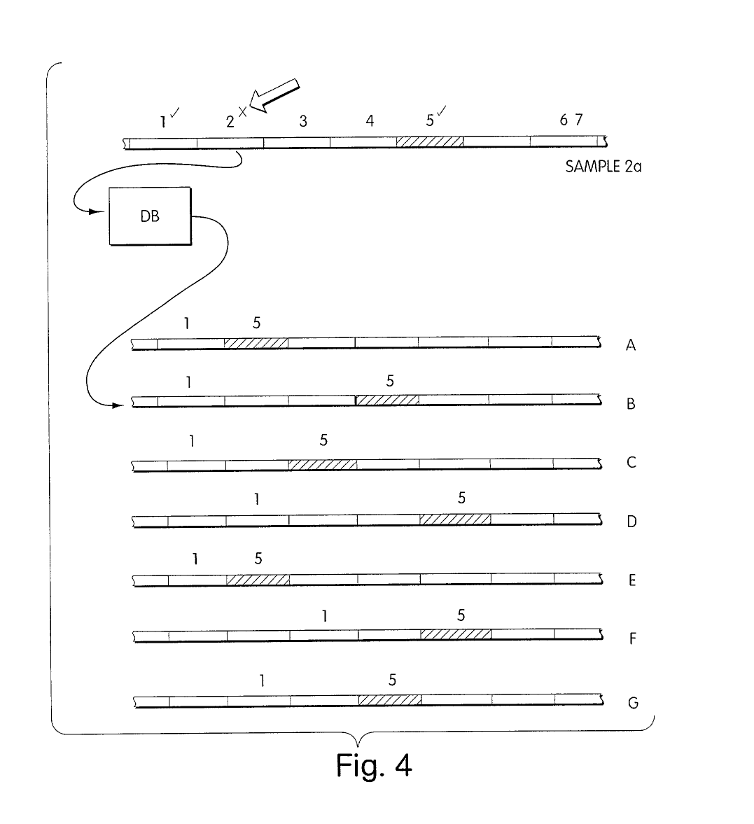

FIGS. 3 and 4 illustrate the procedure for two samples, namely samples 2a and 2b. In the case of sample 2a (FIG. 4), MS/MS data for peptide 5 (as shown by the hatched lines) is searched in the database to reveal candidate proteins A to G, all containing the peptide 5. Then, iterative, MS data for peptide 1 is checked and, in this case, found in all proteins A to G. Then peptide 2 is checked and none of the proteins A to G are found to contain it. In other words, depending on the confidence value used, peptide 2 is not found to the confidence required. For example, when the residual mass of peptide 2 is compared with all the residual masses of the proteins A to G, it may be that no two residual masses match. This may, for example, be the result of a mass spectrometer which does not have the accuracy necessary to provide a close enough measure of the residual mass.

In this case, as shown in FIG. 3, peptide 2 is selected and MS/MS data (as shown by the hatched lines) is recorded by the system and the database searched to find, in this example, candidate proteins A to G. A peptide by peptide comparison of the sample protein against the candidate proteins then finds a match with candidate protein A.

The present technique may be used in a number of ways including, for example, “entry point validation” for pre-screening of proteins prior to protein identification by mass spectrometry. This is done by using the information known about an entry point bait molecule to prepare and guide the mass spectrum experiments to identify the unknown protein expected to bind with the bait molecule.

In this case, the protein entry point/bait or small molecule entry point/bait, protein databases will be searched to compile the list of known binding proteins. This list of known binding proteins will then be expanded by searches (such as those known as “BLAST”) of protein and DNA databases. The compiled list of proteins will then be digested in silico using known enzyme cutting sites, generating a list of peptides for each protein in the compiled database. This list will then be used to guide the mass spectrum experiments.

Thus, a sample may be prepared which includes at least one unknown protein. At least one bait molecule may then be added to the sample, wherein the bait molecule is known to bind with at least one protein. The baited sample is then subjected to the protein analysis as above described, wherein the interactively comparing step includes the step of building a binding protein database according to proteins known to bind with said bait molecule or a consequential molecule thereof. The binding protein database should include data on in silico digests of the list of proteins. The present invention may also be cased to guide and reduce the operations of the mass spectrometer when repeat experiments are necessary which use the same bait or entry point and far differential experiments. In this case, the present technique should provide a reduction of experimental time and allow the MS/MS phase of the mass spectrometer function to be focused on unidentified peptides.

For every entry point or bait the present technique will keep track of the proteins previously identified by the mass spectrometry. It will also generate, by in silico digestion, a list of peptides related to these proteins. This list of peptide masses will be used to guide the next set of mass spectrometry experiments. Any peptides from this list that will be detected by the mass spectrometer will not be selected for MS/MS. Furthermore, an annotation mark will be introduced in the peptide list for every detected peptide. This means that although no MS/MS spectra will be generated the list of annotated peptides that relate to a particular protein will be sufficient to prove the presence of these proteins. The peptides that are not in this list will trigger the MS/MS mode of the mass spectrometer following the above mentioned procedure. This technique will be generally applicable for ESI and MALDI based systems.

### Equivalents

Those skilled in the art will recognize, or be able to ascertain using no more than routine experimentation, numerous equivalents to the specific procedures described herein. Such equivalents are considered to be within the scope of this invention and are covered by the following claims.

1. A method of analyzing a digested protein sample, comprising:
(a) generating an MS data set for the digested sample;
(b) selecting at least one peptide represented in the MS data set;
(c) generating an MS/MS data set for said at least one peptide selected in step (b);
(d) searching a first level protein database to find candidate proteins containing said at least one peptide selected in step (b);
(e) comparing MS data from the digested sample with one or more of the candidate proteins only, to find a match therebetween.

2. A method as defined in claim 1 wherein step (d) includes the step of:
(f) preparing a second level database containing only the candidate proteins, and searching said second database to find candidate proteins containing said at least one peptide selected in step (b).

3. A method as defined in claim 1 wherein step (e) includes the step of:
(g) narrowing the search field in the first level protein database to search only the candidate proteins.

4. A method as defined in claim 1 wherein the first level database includes digest data for each of the candidate proteins.

5. A method as defined in claim 1, further comprising the step of:
(h) generating in silico digest data for at least one of the candidate proteins.

6. A method as defined in claim 4 or 5, wherein step (e) includes the step of:
(i) selecting a peptide from the MS data set;
(j) searching the digest data for the candidate proteins to identify the selected peptide therein; and
(k) recording a match when the selected peptide is found in a candidate protein.

7. A method as defined in claim 1 wherein step (e) includes obtaining another MS data set for the protein sample.

8. A method as defined in claim 6 wherein steps (i), (j) and (k) are repeated until a sufficient number of selected peptides are identified in a candidate protein to declare a match.

9. A method as defined in claim 6 wherein, when a selected peptide of step (i) is not found in any one candidate protein, further comprising the steps of:
(l) generating an MS/MS data set for the selected peptide; and
(m) searching a first level protein database to find candidate proteins according to the selected peptide.

10. A method as defined in claim 1 wherein step (e) is conducted on an online database.

11. A method as defined in claim 1 wherein steps (a) to (d) are carried out in a tandem mass spectrometer (MS/MS).

12. A method as defined in claim 1 wherein steps (a) to (d) are carried out in a mass spectrometer capable of generating MS and MS/MS data.

13. A method as defined in claim 12 wherein steps (a) to (d) are carried out on an ion trap mass spectrometer.

14. A method of analyzing a digested protein sample, comprising the steps of:
(a) generating an MS data set for the digested sample;
(b) selecting a first peptide represented in the data set;
(c) generating an MS/MS data set for the first selected peptide;
(d) searching at least one first level protein database to find at least one candidate protein which, by a predetermined measure, is identified to contain the first selected peptide;
(e) preparing a second level database containing only the candidate proteins of step (d);
(f) selecting a second peptide; and
(g) searching the second level database to find candidates which are identified to contain the selected second peptide; and
wherein, if more than one candidate protein is identified in step (g), further comprising the steps of:
(h) selecting an n^{th} peptide;
(i) searching the second level database to find candidates which are identified to contain the selected n^{th} peptide; and
(j) incrementing n and repeating steps (h), (i), if necessary, until a single candidate protein is identified.

15. A method as defined in claim 14 wherein the step (c) includes the step of narrowing the search field in the first level database.

16. A protein analysis system, comprising:
(a) an MS unit for generating MS data on a digested protein sample;
(b) a selector unit for selecting a first peptide from the digested protein sample;
(c) an MS/MS unit for generating MS/MS data for the first peptide; and
(d) an identification unit for identifying the protein sample, the identification unit comprising:
(I) a search station operable in a first phase for searching at least one first level database to identify candidate proteins containing the first peptide;
(II) a memory station for storing art least one second level database containing only the candidate proteins;
(III) the search station being operable in a second phase to find a single target candidate protein by comparing the MS data from the digested protein sample with MS data for the candidate proteins.

17. A system as defined in claim 16 wherein the MS/MS unit is in tandem with the MS unit.

18. A protein analysis system, comprising:
(a) an MS unit for generating mass spectrum data on a digested protein sample;
(b) selection means for selecting a peptide from the digested protein sample;
(c) an MS/MS unit for generating mass spectrum data for the selected peptide; and
(d) an identification unit for identifying the protein sample, the identification unit comprising a general purpose computer programmed to carry out the steps of,
(e) searching at least one first level database to identify candidate proteins containing the first peptide;
(f) storing at least one second level database containing only the candidate proteins;
(g) searching the second level database to identify a single target candidate protein by comparing the MS data from the digested protein sample with MS data for the candidate proteins.

19. A computer program product recorded on a computer-readable medium and including the computer executable steps of:
(a) initiating a computer data input to receive MS data of a digested protein sample;
(b) selecting one peptide from the MS data;
(c) initiating a computer data input to receive MS/MS data of the selected peptide;
(d) initiating a search of a protein database to find candidate proteins which, by some measure of confidence, contain the selected peptide;
(e) comparing the peptides of the digested protein with the candidate proteins in order to identify a candidate sharing a sufficient predetermined number of peptides to declare a match; and
(f) generating an output to report the match.

20. A method of protein analysis, comprising the steps of:
(a) selecting a peptide from MS data for a digested protein sample;
(b) recording MS/MS data for the selected peptide;
(c) initiating a search of a protein database to find candidate proteins which, by some measure of confidence, contain the selected peptide; and
(d) iteratively comparing the peptides of the digested protein with the candidate proteins in order to identify a candidate sharing a sufficient predetermined number of peptides to declare a match.

21. A method of protein analysis, comprising:
(a) preparing a sample comprising at least one unknown protein;
(b) adding to the sample at least one bait molecule;
(c) subjecting the baited sample to the method of claim 20, wherein before step (c), the method includes the step of building a binding protein database according to proteins known to bind with said bait molecule or a consequential molecule thereof

22. A method as defined in claim 21 wherein step (c) includes the steps of:
(d) assembling a list of proteins known to bind with said bait molecule or a consequential molecule thereof;
(e) conducting an in silico digestion of the list of proteins to form said binding protein database.

23. A method of protein analysis, comprising:
(a) preparing a list of known proteins and conducting an in silico digestion of the list of proteins to form a peptide database;
(b) providing a digested protein sample;
(c) recording MS data for the digested protein sample;
(d) selecting a first peptide from the digested protein sample;
(e) recording MS/MS data for the first selected peptide;
(f) initiating a search in the peptide database to find candidate proteins which, by a predetermined confidence value, contain the first selected peptide;
(g) selecting a second peptide; and
(h) comparing the MS data of the second peptide with the candidate proteins in order to find candidate proteins which contain both the first and second selected peptides.

24. A method as defined in claim 23 wherein, when more than one match has been found in step (h), further comprising the step of:
(i) selecting another peptide and repeating step (h).

25. A method as defined in claim 23 wherein, when a match is not found in step (h), further comprising the steps of:
(j) recording MS/MS data for the second selected peptide;
(k) initiating a search in the peptide database to find candidate proteins which, by a predetermined confidence value, contain the second selected peptide.
